(19) United States
(12) Patent Application Publication
(10) Pub. No.: US 2009/0226898 A1
(43) Pub. Date: Sep. 10, 2009

(54) Mitochondrial markers of ischemia

(75) Inventor: Paul Sheard, Northants (GB)

Correspondence Address: Steptoe & Johnson LLP, 1330 Connecticut Avenue, NW, Washington DC, DC, 20036, US

(73) Assignee: Inverness Medical Switzerland GMBH, Zug (CH)

(21) Appl. No.: 11/988,507

(22) PCT Filed: Jul. 13, 2006
(86) PCT No.: PCT/IB2006/001934

§ 371 Date: Apr. 28, 2008

Related U.S. Application Data
(60) Provisional application No. 60/698,934, filed on Jul. 14, 2005.
(60) Provisional application No. 60/700,316, filed on Jul. 19, 2005.
(60) Provisional application No. 60/751,310, filed on Dec. 19, 2005.

(51) Int. Cl. C12Q 1/68; C12Q 1/34; C12M 1/34

(52) U.S. Cl. 435/6; 435/18; 435/287.2

(57) ABSTRACT

Damage to tissue, such as ischemic damage, can cause the release of mitochondrial markers. The released markers can be detected in a sample taken from a subject, indicating that the subject has suffered damage.

### CLAIM OF PRIORITY

This application claims priority to U.S. Application No. 60/698,934, filed Jul. 14, 2005, U.S. Application No. 60/700,316, filed Jul. 19, 2005, and U.S. Application No. 60/751,310, filed Dec. 19, 2005, each of which is incorporated by reference in its entirety.

### TECHNICAL FIELD

This application relates to markers of ischemia, particularly to mitochondrial markers of cardiac damage.

### BACKGROUND

Cardiac markers serve an important role in the early detection and monitoring of cardiovascular disease. Markers of disease are typically substances found in a bodily sample that can be easily measured. The measured amount can correlate to underlying disease pathophysiology, presence or absence of a current or imminent cardiac event, probability of a cardiac event in the future. In patients receiving treatment for their condition, the measured amount will also correlate with responsiveness to therapy. Markers can include elevated levels of blood pressure, cholesterol, blood sugar, homocysteine and C-reactive protein (CRP). However, current markers, even in combination with other measurements or risk factors, do not adequately identify patients at risk, accurately detect events (i.e., heart attacks), or correlate with therapy. For example, half of patients do not have elevated serum cholesterol or other traditional risk factors.

Myocardial ischemia can be a main cause of the acute coronary syndromes (ACS), a continuum of disease that spans from unstable angina (characterized by reversible cardiac ischemia) to myocardial infarction with large areas of necrosis. Myocardial ischemia can result from thrombus formation after plaque rupture in a coronary artery. The acute coronary syndromes represent a complex and heterogeneous physiological condition. Although remarkable therapeutic and technological advances over the past 20 years have reduced the in-hospital mortality of acute myocardial infarction, this progress has been limited to patients who display ST-elevation on their electrocardiogram (ECG). ST-elevation is an indicator of myocardial infarction, and treatment within 12 hours of symptoms onset will improve the outcome. However, only about 50% of myocardial infarction patients have diagnostic ECG changes. The remaining patients must be observed for clinical monitoring signs and biochemical markers such as cardiac troponin T or I.

Cardiac troponin has become the cornerstone for diagnosis of myocardial infarction. Markers such as CK-MB and myoglobin can be useful for assessment and risk stratification of suspected ACS patients. Compelling evidence indicates that an elevated cardiac troponin can identify high-risk ACS patients that benefit from treatment with antiplatelet agents including; inhibitors of the glycoprotein IIb/IIIa platelet receptor (such as abciximab, eptifibatide, lamifiban and tirofiban), non-specific COX inhibitors (such as acetylsalycilic acid) and ADP receptor antagonists (such as clopidogrel and ticlopidine). However, troponin, CK-MB and myoglobin are markers of necrosis and therefore offer no information regarding myocardial ischemia that occurred before cell death. A test that can accurately detect the presence or absence of myocardial ischemia allowing treatment decisions to be made at an earlier stage of the ACS continuum will have significant clinical utility. Further, therapeutic options specifically targeting this early stage of ACS has the potential to significantly improve patient prognosis.

### SUMMARY

Eukaryotic cells contain mitochondria, organelles that produce energy for the cell. In multicellular organisms, different types of cells can have different numbers of mitochondria. For example, in animals, muscle cells can have a high number of mitochondria, in order to provide energy for muscle function. Injury to cells, tissues or organs can cause disruption of mitochondria and the release of their contents.

Muscle cells (e.g., myocardial cells) contain a high proportion of muscle proteins (e.g., actin, myosin, troponin) and mitochondria devoted to producing energy to drive muscle contraction. Damage to myocardial cells, such as occurs when the myocardium is subject to ischemia, can cause the contents of the cells to be released. The cellular contents can be detected in other bodily samples (for example, in the blood). In particular, mitochondria can be disrupted, and the contents of the mitochondria can be detected elsewhere. These detectable components of mitochondria can be diagnostic of health, for example, ischemia or cardiac health including cardiac damage. A mitochondrial marker (i.e., a peptide normally localized in mitochondria and including at least two amino acid residues) can be one such component diagnostic of health.

In one aspect, a method for monitoring health of a mammalian subject includes identifying a mitochondrial marker in a sample obtained from a subject and associating the mitochondrial marker with a status of health of the subject.

Monitoring health can include detecting, screening, diagnosing, monitoring, or managing therapy of acute coronary syndromes. In other circumstances, monitoring health can include detecting, screening, diagnosing, monitoring, or managing therapy of chronic angina. In other circumstances, monitoring health can include detecting, screening, diagnosing, monitoring, or managing therapy of ischemia and the subject is in a patient suffering from stroke. In yet other circumstances, monitoring health can include detecting, screening, diagnosing, monitoring, or managing therapy of ischemia and the subject is in a patient suffering from heart failure.

Associating the mitochondrial marker with a status of health can include assessing the cardiac health of the subject, the neurological health of the subject, the pulmonary health of the subject, or a treatment protocol for the subject.

The method can include obtaining the sample from the patient before cardiac surgery, exercise treadmill, or pharmacologic stress testing. The method can include obtaining the sample from the patient during cardiac surgery, exercise treadmill, or pharmacologic stress testing. The method can include obtaining the sample from the patient after cardiac surgery, exercise treadmill, or pharmacologic stress testing. The method can include obtaining the sample at any or each of these times.

The sample can be blood, plasma, or serum. The sample can be used directly in the method, or can be processed to prepare the sample for the method. In the method, identifying a mitochondrial marker can include determining a level of the mitochondrial marker in the sample.

In another aspect, a system for monitoring health includes a cartridge including a sample port and a first assay, wherein the first assay recognizes a mitochondrial marker, and a cartridge reader including a detector configured to measure a level of the mitochondrial marker recognized by the assay. The device can be configured to provide an output to a patient. The cartridge can further include a second assay. The cartridge reader can include a detector configured to measure a second assay. The second assay can recognize a necrosis marker or an ischemia marker. The first assay can include a ligand for the mitochondrial marker.

In another aspect, a cartridge for monitoring health includes a cartridge including a sample port and a first assay, wherein the first assay recognizes a mitochondrial marker. The cartridge can include a second assay, which can a necrosis marker or an ischemia marker. The first assay can include a ligand for the mitochondrial marker.

The mitochondrial marker can be a polypeptide encoded by nuclear DNA, or a polypeptide encoded by mitochondrial DNA. When encoded by nuclear DNA, a polypeptide can be transported to the mitochondrion after translation. The mitochondrial marker can be a subunit of NADH dehydrogenase, a subunit of cytochrome c oxidase, a subunits of F0F1ATPase, or cytochrome b. The mitochondrial marker can be a formyl peptide receptor (FPR) ligand. An FPR ligand can optionally include an N-formyl group, for example, N-formyl methionine. The FPR ligand can be derived from a mitochondrial marker. For example, the FPR ligand can be a breakdown product (e.g., a hydrolysis product) of a mitochondrial marker. The mitochondrial marker can be a mitochondrial DNA, a mitochondrial RNA, an N-formyl polypeptide, a peptide encoded by mitochondrial DNA, or an FPR ligand, or a caspase. In certain circumstances, the mitochondrial marker can be a regulatory marker, such as, for example, cytochrome c, apoptosis-inducing factor, apoptotic protein activating factor-1, second mitochondria-derived activator of caspases, direct IAP-binding protein, serine protease omi/HtrA2, or endonuclease G. Detection of a predetermined amount of the mitochondrial marker can be indicative of the status of health of the subject, for example, of cardiac ischemia.

The method can further include identifying a necrosis marker in the sample, such as, for example, a troponin, CK-MB, myoglobin, or fatty acid binding protein. In certain circumstances, the method can further include identifying an ischemia marker in the sample, such as, for example, ischemia modified albumin, a fatty acid, whole blood choline, lipoprotein-associated phospholipase, or an oxidised lipid.

The sample can be contacted with a ligand for the mitochondrial marker. The ligand can be an antibody, an antibody fragment, a modified antibody, chimeric antibody, soluble receptor, or aptamer. For example, the ligand can be a formyl peptide receptor, a formyl peptide receptor sub-unit, a fragment of a formyl peptide receptor, or an encoded sequence of binding region of a formyl peptide.

The subject can be a human subject, or a non-human subject such as, for example, a bird, a mouse, a rat, a rabbit, a pig, a sheep, a goat, a cow, or another mammal.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 is a schematic diagram depicting metabolic pathways in muscle tissue.

FIG. 2 is a schematic diagram depicting the link between reactive oxygen species, mitochondrial complex defects, and mitochondrial dysfunction.

FIG. 3 is a schematic diagram of the arrangement of subunits in mitochondrial complex I.

FIG. 4 is an illustration of a cartridge reader.

### DETAILED DESCRIPTION

The health of a subject can be monitored by identifying a mitochondrial marker in a sample obtained from a subject and associating the mitochondrial marker with a status of health of the subject. The status of health can be monitored by evaluating the ischemic events being experienced by the subject. The ischemia can occur as supply ischemia or demand ischemia. The ischemia can occur in a tissue such as heart, lung, muscle, or brain, thus permitting the cardiac, pulmonary, muscular or neurological health of the subject to be monitored.

For example, an ischemic stroke occurs when an artery to the brain is blocked. Ischemic stroke can further be divided into two main types: thrombotic or embolic.

A thrombotic stroke occurs when diseased or damaged cerebral arteries become blocked by the formation of a blood clot within the brain. Clinically referred to as cerebral thrombosis or cerebral infarction, this type of event is responsible for almost 50% of all strokes. Cerebral thrombosis can also be divided into an additional two categories that correlate to the location of the blockage within the brain: large-vessel thrombosis and small-vessel thrombosis. Large-vessel thrombosis occurs when the blockage is in one of the brain's larger blood-supplying arteries such as the carotid or middle cerebral, while small-vessel thrombosis involves one (or more) of the brain's smaller, yet deeper penetrating arteries.

An embolic stroke is also caused by a clot within an artery, but in this case the clot (or emboli) was formed somewhere other than in the brain itself. Often from the heart, these emboli will travel the bloodstream until they become lodged and can not travel any further. This naturally restricts the flow of blood to the brain and results in almost immediate physical and neurological deficits.

One consequence of ischemia is damage and/or dysfunction at the mitochondrial energy level resulting in leakage of markers from the mitochondrion into the cytosol and into circulation in the subject. In addition, ischemia can trigger apoptosis via the mitochondria pathway of apoptosis. See, Haunstetter, Armin et al., “Basic Mechanisms and Implications for Cardiovascular Disease,” Apoptosis, Vol. 82, pp. 1111-1129, 1998; and Bennett, Martin R., “Apoptosis in the Cardiovascular System,” Heart, Vol. 87, pp. 480-487, 2002, each of which is incorporated by reference in its entirety. Moreover, the energetics in heart failure indicate that an increase in apoptosis which drives remodelling of the heart. See, for example, Fujii, Nozomu et al., “Saturated glucose uptake capacity and impaired fatty acid oxidation in hypertensive hearts before development of heart failure,” AJP-Heart, Vol. 287, pp. 760-766, 2004; van Bilsen, Marc, “‘Energenetics’ of Heart Failure,” Annals New York Academy of Sciences, Vol. 1015, pp. 238-249, 2004; van Bilsen, Marc, “Metabolic remodelling of the failing heart: the cardiac burn-out syndrome?,” Cardiovascular Research, Vol. 61, pp. 218-226, 2004; Mak, Suzanne et al., “The Oxidative Stress Hypothesis of Congestive Heart Failure: Radical Thoughts,” Chest, Vol. 120, pp. 2035-2046, 2001; Sambandam, Nandakumar et al., “Energy Metabolism in the Hypertrophied Heart,” Heart Failure Reviews, Vol. 7, pp. 161-173, 2002; and Ventura-Clapier, Renee et al., “Energy metabolism in heart failure,” J. Physiol., Vol. 555, pp. 1-13, 2003, each of which is incorporated by reference in its entirety. Thus, markers for apoptosis can be detectable markers for ischemic events.

The mitochondrial marker can be used to assist in the detection, diagnosis, prognosis, or therapy management of acute coronary syndromes (ACS) and ischemic events such as, for example, stroke. For example, the presence of a mitochondrial marker, particularly above a threshold level, can be indicative of ischemia arising through supply ischemia associated with a thrombus. In other circumstances, the mitochondrial marker can be used to assist in the detection, diagnosis, prognosis, or therapy management of chronic angina. For example, the presence of a mitochondrial marker, particularly above a threshold level, can be indicative of ischemia arising through supply ischemia not associated with a thrombus. In other circumstances, the mitochondrial marker can be used to assist in the detection, diagnosis, prognosis, or therapy management of ischemia in a heart failure patient. For example, the presence of a mitochondrial marker, particularly above a threshold level, can be indicative of ischemia arising through demand ischemia. In other circumstances, the mitochondrial marker can be used to assist in the detection, diagnosis, prognosis, or therapy management of ischemia in a patient before, during, and after cardiac surgery. Links between ischemia, myocardial inflammation, and apoptosis during and after cardiac surgery and tissue markers for apoptosis are described, for example, in Anselmi, Amedeo et al., “Myocardial ischemia, stunning, inflammation, and apoptosis during cardiac surgery: a review of evidence,” European Journal of Cardio-thoracic Surgery, Vol. 25, pp. 304-311, 2004, which is incorporated by reference in its entirety.

A subject with pre-heart failure or at risk for heart failure or heart failure or an early stage of decompensated heart failure can be managed in the home or a non-hospital setting. To help such subjects monitor the likelihood of cardiovascular events occurring or therapeutically manage their condition, a means is provided to detect or to monitor the subject's health or condition. The method for monitoring the health of a subject can further include measuring the level of a marker in the sample and associating a level of the second marker with the status of cardiac health. In other circumstances, the mitochondrial marker can be used to assist in the detection, diagnosis, or prognosis of ischemia during exercise treadmill or pharmacologic stress testing.

An assay for the mitochondrial marker can be combined with a second assay to detect a necrosis marker, such as, for example, myoglobin, troponin, creatine kinase MB (CK-MB) or fatty acid binding protein (FABP), or an ischemia marker such as, for example, ischemia modified albumin, a fatty acid, whole blood choline, lipoprotein-associated phospholipase, an oxidised lipid, or an ischemic stroke marker such as, for example, neuron specific enolase (nse), S100beta to evaluate the health of a subject. The assay can further include the detection of a thrombotic marker or platelet activation marker such as (fe) thromboxane, sCD40L, sP-selectin, soluble fibrin, thrombus precursor protein or d-dimer. The presence and level of each of these markers can be used to evaluate a risk of heart failure, diagnose early or late stages of decompensated heart failure, heart failure, or risk of heart failure, to prognose adverse outcomes in the subject, or to monitor an effect of therapy administered to the subject. Ischemia markers tend to have a faster release profile than necrosis markers (e.g. myoglobin or fatty acid binding protein (H-FABP)), and can provide additional information relating to the health of the subject.

Many of other tests and procedures for accurately and successfully monitoring, diagnosing, managing and treating health, particularly cardiac health, are complex, expensive and available only at a hospital or other health-care settings. Methods for patients to manage or to monitor health at home or otherwise outside a health-care setting can be even less successful. Advantageously, the measurement of a mitochondrial marker can be useful as a more sensitive and accurate means for monitor health of a subject at the early stages of ischemia, and during later stages of ischemic events.

Health can also be monitored by observing a change in intraindividual variability, observing a change in absolute or relative level from a baseline level established within an individual or observing a positive or negative trend over time. For example, health of a subject can be monitored by measurement of one or more mitochondrial marker levels in combination with measurement of a subject's vital signs, such as weight, temperature, heart rate, breathing rate, blood pressure, and blood oxygen saturation (pulse oximetry). The levels of a mitochondrial marker may also be measured in combination with chest X-ray, electrocardiography, echocardiography, radionuclide imaging and dilutional analysis.

The mitochondrial marker is a biological marker released from a cell about to, or in the process of enduring an ischemic event. The mitochondrial marker is released from the mitochondrion of a cell, or triggers an intracellular event that causes release of another chemical entity from the cell. The mitochondrial marker can be a mitochondrial nucleic acid, such as a mitochondrial DNA (mtDNA) or mitochondrial RNA (mtRNA). The mitochondrial marker can be a polypeptide encoded by nuclear DNA, or a polypeptide encoded by mitochondrial DNA. When encoded by nuclear DNA, a polypeptide can be transported to the mitochondrion after translation. The mitochondrial marker can be an isoform, post-translational product, deletion/degradation product, or other derivative of a mitochondrial polypeptide. For example, the mitochondrial marker can optionally have a N-formyl-methionine residue at its N-terminus.

A mitochondrial nucleic acid, generally, is a nucleic acid localized in the mitochondria, such as mtDNA or mtRNA (including messenger RNA, transfer RNA, and ribosomal RNA). The mitochondrial nucleic acid can have a sequence that encodes a mitochondrial polypeptide, a regulatory sequence, or another sequence. The mitochondrial nucleic acid can have a sequence that is complementary (i.e., capable of hybridizing with) a sequence that encodes a mitochondrial polypeptide, a regulatory sequence, or another sequence. Listings of human mitochondrial DNA sequences can be found, for example, at Mitomap (www.mitomap.org). Descriptions of mitochondrial polypeptides (both nuclear and mitochondrially encoded) can be found, for example in the Human Mitochondrial Protein Database (http://bioinfo.nist.gov:8080/examples/servlets/index.html).

The mitochondrial marker can be a member of one of the following families: caspases, pro-/anti-apoptotic regulators, or oxidoreductases. More specifically, the mitochondrial marker can be a subunit of NADH dehydrogenase, a subunit of cytochrome c oxidase, a subunits of F0F1ATPase, cytochrome b, a formyl peptide receptor (FPR) ligand, an N-formyl polypeptide, a caspase, a regulatory marker, such as, for example, cytochrome c, apoptosis-inducing factor, apoptotic protease activating factor, apoptotic protein activating factor-1, a second mitochondria-derived activator of caspase, a member of the bcl-2 family, direct IAP-binding protein, serine protease omi/HtrA2, or endonuclease G. The mitochondrial marker can be a nucleic acid that encodes any of the above polypeptides, or is capable of hybridizing to a nucleic acid that encodes any of the above polypeptides.

The mitochondrial marker can be a mitochondrial DNA, a mitochondrial RNA, or a mitochondrial encoded protein, such as a subunit of NADH dehydrogenase, a subunit of cytochrome c oxidase, a subunit of F0F1ATPase, or cytochrome b, each of which are encoded by mitochondrial DNA and synthesized in the organelle. Mitochondrial encoded protein markers can include the full length polypeptide and resultant fragments from processing and degradation.

Subunits of NADH dehydrogenase include seven subunits (ND1, ND2, ND3, ND4, ND4L, ND5, and ND6). Complex I (NADH-ubiquinone reductase) is a multimeric assembly of 7 mitochondrial-encoded subunits (ND subunits) and at least 36 nuclear-encoded subunits. Complex I is the first step in the electron transport chain of mitochondrial oxidative phosphorylation (OXPHOS) and is located within the mitochondrial inner membrane. It accepts electrons from NADH and transfers them, through a series of electron carriers, to ubiquinone (Coenzyme Q10). The internal electron carriers of complex I include flavin mononucleotide (FMN) and 6 iron-sulfur clusters designated N-1a, N-1b, N-2, N-3, N-4, and N-5. See Ohnishi, T.: Mitochondrial iron-sulfur flavohydrogenases. In: Capaldi, R. A.: Membrane Proteins in Energy Transduction. New York: Dekker (pub.) 1979; Ragan, C. I.: Structure of NADH-ubiquinone reductase (Complex I). Curr. Top. Bioenerg. 15: 1, 1987, each of which is incorporated by reference in its entirety. Complex I can be subdivided into 3 main fractions: the flavoprotein fragment, the iron-protein fragment, and the hydrophobic protein fragment. See Ragan, C. I.: Structure of NADH-ubiquinone reductase (Complex I). Curr. Top. Bioenerg. 15: 1, 1987, which is incorporated by reference in its entirety.

Subunits of cytochrome c oxidase include COI, COII, and COIII. Cytochrome c oxidase includes three mitochondrial DNA (mtDNA) encoded subunits (MTCO1, MTCO2, MTCO3) of respiratory Complex IV. Complex IV is located within the mitochondrial inner membrane and is the third and final enzyme of the electron transport chain of mitochondrial oxidative phosphorylation.

Subunits of F0F1ATPase include ATPase 6 and ATPase 8. Complex V (ATP synthase) of the mitochondrion includes 10-16 subunits encoded by nuclear DNA and 2 subunits (ATPase 6 and ATPase 8) encoded by mtDNA. Subunit 6 of mitochondrial ATP synthase (complex V) is encoded by nucleotides 8527-9207 of the mitochondrial genome whereas Subunit 8 of mitochondrial ATP synthase (complex V) is encoded by nucleotides 8366-8572 of the mitochondrial genome.

Cytochrome b (MTCYB) is the only mitochondrial DNA (mtDNA) encoded subunit of respiratory Complex III. Complex III is located within the mitochondrial inner membrane and is the second enzyme in the electron transport chain of mitochondrial oxidative phosphorylation. It catalyzes the transfer of electrons from ubiquinol (reduced Coenzyme Q10) to cytochrome c and utilizes the energy to translocate protons from inside the mitochondrial inner membrane to outside.

Other mitochondrial markers can be used. Caspases are triggered by cytochrome c release and can be used to measure or detect ischemia as part of the full cascade of proteolysis and cell death. Caspases include caspase initiators and caspase effectors. Caspase initiators can be caspase-2, caspase-8, caspase-9, or caspase-10. Caspase effectors can be caspase-3, caspase-6, or caspase-7. Biomarkers of caspases can include the full length inactive caspase (for example, pro-caspase-9), a processed caspase (for example, caspase-9), and resultant fragments, such as a product of the action of an initiator caspase on an effector caspase, for example, the action of caspase-9 on caspase-3 or caspase-7, resulting in a low and a high molecular weight fragment. See, for example, Cohen, Gerald M., “Caspases: the executioners of apoptosis,” Biochem. J., Vol. 326, pp. 1-16, 1997, which is incorporated by reference in its entirety. Caspase 3, an apoptosis-related cysteine protease, or CASP3, exists as an inactive precursor of 32 kD until converted proteolytically to a 20 kD and 10 kD heterodimer when cells are signaled to die. See, for example, Feng, Jun et al., “Improved Profile of Bad Phosphorylation and Caspase 3 Activation After Blood Versus Crystalloid Cardioplegia,” Ann. Thorac. Surg., Vol. 77, pp. 1384-1390, 2004, which is incorporated by reference in its entirety. Caspase 9, an apoptosis-related cysteine protease, or CASP9, can be critical for cytochrome c-dependent apoptosis and normal brain development. Caspase 9 participates in caspase 3 activation in vitro. See, for example, Krajewski, Stanislaw, et al., “Release of caspase-9 from mitochondria during neuronal apoptosis and cerebral ischemia,” Proc. Natl. Acad. Sci. USA, Vol. 96, pp. 5752-5757, 1999; and Zou, Hua et al., “An APAF-1-Cytochrome c Multimeric Complex Is a Functional Apoptosome That Activates Procaspase-9,” The Journal of Biological Chemistry, Vol. 274, No. 17, pp. 11549-11556, 1999, each of which is incorporated by reference in its entirety.

Regulatory markers include pro-apoptotic and anti-apoptotic regulators. The regulatory markers can include regulatory DNA or RNA sequences, e.g., a promoter sequence, a repressor sequence, or other noncoding sequence. The regulatory markers can include regulatory polypeptides or assembles of polypeptides that activate or inhibit the apoptotic process. A regulatory marker can include the bcl-2 family, apaf (apoptotic protease activating factor), cytochrome c, iap (inhibitor of apoptosis), aif (apoptosis inducing factor), smac (second mitochondria-derived activator of caspases) or diablo (direct IAP-binding protein), or the serine protease omi/HtrA2, or endonuclease G. Regulatory markers can be polypeptides or assembles that can include a full length regulatory polypeptide, a processed polypeptide. For example, smac is proteolytically cleaved upon import into the mitochondria leaving a low and high molecular weight component, and endonuclease G is processed to remove the mitochondrial localisation signal.

For example, the bcl-2 family of proteins consists of both inhibitors and promoters of programmed cell death. Bcl-2 is anti-apoptotic, and prevents the activation of caspase 3. Bad is pro-apoptotic. Phosphorylation of Bad inhibits binding to and inactivation of anti-apoptotic Bcl-2 and phosphorylation of Bad increases in hearts exposed to cardioplegic ishemic and reperfusion injury. See, for example, Feng, Jun et al., “Improved Profile of Bad Phosphorylation and Caspase 3 Activation After Blood Versus Crystalloid Cardioplegia,” Ann. Thorac. Surg., Vol. 77, pp. 1384-1390, 2004, which is incorporated by reference in its entirety. The proapoptotic BAX protein induces cell death by acting on mitochondria, protein partner of bcl-2. See, for example, Oltvai, Z. N.; Milliman, C. L.; Korsmeyer, S. J. “Bcl-2 heterodimers in vivo with a conserved homolog, Bax, that accelerates programmed cell death.” Cell 74: 609-619, 1993, which is incorporated by reference in its entirety.

Apoptotic protease activating factor (Apaf) is a 130-kD protein that participates in the cytochrome c dependent activation of caspase-3. See, for example, Zou, Hua et al., “An APAF-1-Cytochrome c Multimeric Complex Is a Functional Apoptosome That Activates Procaspase-9,” The Journal of Biological Chemistry, Vol. 274, No. 17, pp. 11549-11556, 1999, which is incorporated by reference in its entirety.

Cytochrome c is released and the uncoupled from mitochondria, for example, by oxidative stress activating the c-JNK pathway which in turns causes the release cytochrome c by selective permeabilization of the outer mitochondrial membrane. Mitochondrial signaling of apoptosis via cytochrome c release can be preserved in cells lacking mitochondrial DNA. See, for example, Kuznetsov Andrey V. et al., “Mitochondrial defects and heterogeneous cytochrome c release after cardiac cold ischemia and reperfusion,” Am. J. Physiol Heart Circ. Physiol., 10:1152, 2003; Aoki, Hiroki et al., “Direct Activation of Mitochondrial Apoptosis Machinery by c-Jun N-terminal Kinase in Adult Cardiac Myocytes,” The Journal of Biological Chemistry, Vol. 277, No. 12, pp. 10244-10250, 2002; Jiang, Shunai et al., “Cytochrome c-mediated Apoptosis in Cells Lacking Mitochondrial DNA,” The Journal of Biological Chemistry, Vol. 274, No. 42, pp. 29905-29911, 1999, each of which is incorporated by reference in its entirety.

Inhibitor of apoptosis (IAP) can include one of three human IAPs-XIAP, c-IAP-1, c-IAP-2 which bind procaspase 9 and prevent activation of caspase 9. IAP can also bind and inhibit active caspases. See, for example, Deveraux, Q. L. and Reed, J. C. (1999), which is incorporated by reference in its entirety. IAP family of proteins are suppressors of apoptosis. See, Genes Dev. 13, 239-252, which is incorporated by reference in its entirety

Apoptosis inducing factor (AIF) promotes apoptosis by translocating to the nucleus from the mitochondrial intermembrane space, where it binds to DNA and initiates a caspase independent chromatin condensation. Studies have suggested that AIF is a major factor in caspase independent neuronal death. See, for example, Candé, Céline et al., “Apoptosis-inducing factor (AIF): key to the conserved caspase-independent pathways of cell death?,” Journal of Cell Science, Vol. 115, pp. 4727-4734, 2002; Aoki, Hiroki et al., “Direct Activation of Mitochondrial Apoptosis Machinery by c-Jun N-terminal Kinase in Adult Cardiac Myocytes,” The Journal of Biological Chemistry, Vol. 277, No. 12, pp. 10244-10250, 2002, each of which is incorporated by reference in its entirety.

Second mitochondria-derived activator of caspases (smac), also known as Diablo (direct IAP-binding protein), promotes caspase activation in the cytochrome c/Apaf-1/caspase 9 pathway. Specifically, smac promotes caspase 9 activation by binding to IAP and removing inhibitory activity. Smac is a mitochondrial protein that is released into cytosol when cells undergo apoptosis. See, for example, Du, Chunying et al., “Smac, a Mitochondrial Protein that Promotes Cytochrome c-Dependent Caspase Activation by Eliminating IAP Inhibition,” Cell, Vol. 102, pp. 33-42, 2000, which is incorporated by reference in its entirety.

Serine protease omi/HtrA2 is released from mitochondria and inhibits the function of XIAP by direct binding in a way similar to smac. See, for example, Suzuki, Y. et al. “A serine protease, HtrA2, is released from the mitochondria and interacts with XIAP, inducing cell death.” Molec. Cell 8: 613-621, 2001, which is incorporated by reference in its entirety.

Endonuclease G is a nuclease specifically activated by apoptotic stimuli. Endonuclease G can induce nucleosomal fragmentation of DNA in fibroblast cells from embryonic mice lacking DFF45. See, for example, Li, L et al., “Endonuclease G is an apoptotic DNase when released from mitochondria.” Nature 412: 95-99, 2001, which is incorporated by reference in its entirety.

The presence or level of a mitochondrial marker can be identified using a ligand. The ligand can include, for example, an antibody, a modified antibody, a chimeric antibody, soluble receptor, aptamer, or other species capable of binding to a mitochondrial marker. An aptamer is a single- or double-stranded DNA or single-stranded RNA molecules that recognize and bind to a desired target molecule by virtue of their shapes. See, e.g., PCT Publication Nos. WO 92/14843, WO 91/19813, and WO 92/05285, each of which is incorporated by reference in its entirety. The ligand can be detectably labeled, for example with a fluorescent dye, colored material, or radioactive isotope. When the marker is a nucleic acid, the ligand can be a nucleic acid capable of hybridizing with the marker, e.g., the ligand can have a sequence complementary to the marker sequence. Alternatively, a nucleic acid marker can be amplified, for example, with a polymerase chain reaction, to increase the amount of nucleic acid available for detection. The amplified product can be detected by, for example, a labeled probe, or a dye that binds to a double-stranded, but not single-stranded, nucleic acid.

The sample DNA can be amplified before detection. The amplification of DNA may also occur in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, other available DNA polymerases, polymerase muteins, reverse transcriptase, ligase, and other enzymes, including heat-stable enzymes (i.e., those enzymes that perform primer extension after being subjected to temperatures sufficiently elevated to cause denaturation). Suitable enzymes will facilitate combination of the nucleotides in the proper manner to form primer extension products that are complementary to each nucleotide strand. Generally, the synthesis will be initiated at the 3′ end of each primer and proceed in the 5′ direction along the template strand, until synthesis terminates, producing molecules of different lengths. There may be amplification agents, however, that initiate synthesis at the 5′ end and proceed in the other direction, using the same process as described above. In any event, the method of the invention is not to be limited to the embodiments of amplification described herein. The polymerase chain reaction (PCR) described in U.S. Pat. Nos. 4,683,202 and 4,683,195 (each of which is incorporated by reference in its entirety) can be used to detect a polynucleotide marker. Many polymerase chain methods are known to those of skill in the art and may be used in the method of the invention.

Amplification products can optionally be radioactively labeled or fluorescently labeled to facilitate detection. For direct detection of amplification products, any label may be employed which provides an adequate signal. Other labels include ligands, which can serve as a specific binding pair member for a labeled ligand, and the like.

For example, the presence or level of a mitochondrial marker in a sample can be measured by quantifying the amount of the marker in the sample as a whole molecule, as fragments of the marker, or by measuring the marker's activity in the sample or a derivative of the sample. Many markers are polypeptides. Fragments of the markers can be measured using a fragment that has an amino acid sequence which is unique to the marker in question. The fragment may be as few as 6 amino acids, although it may be 7, 8, 9, 10, 11, 12, 13, 14, 15 or more amino acids.

The N- or C-terminus of a polypeptide marker can be modified, such as with a formyl group on the N-terminus. The C-terminus can be modified with the addition of a Cys residue for conjugation purposes. The additional peptide sequence can be modified, (for example, glycosylated, phosphorylated, biotinylated, modified with a hydrophobic group (e.g., myristoylated or geranylgeranylated), or other peptide modification. If the polypeptide is synthetic, the modification can include, for example, a colored or fluorescent group, or a poly(ethylene glycol) group.

In general, an amino acid residue of the polypeptide can be replaced by another amino acid residue in a conservative substitution. Examples of conservative substitutions include, for example, the substitution of one non-polar (i.e., hydrophobic) residue such as isoleucine, valine, leucine or methionine for another non-polar residue; the substitution of one polar (i.e. hydrophilic) residue for another polar residue, such as a substitution between arginine and lysine, between glutamine and asparagine, or between glycine and serine; the substitution of one basic residue such as lysine, arginine or histidine for another basic residue; or the substitution of one acidic residue, such as aspartic acid or glutamic acid for another acidic residue. In an conservative substitution, an amino acid residue can be replaced with an amino acid residue having a chemically similar side chain. Families of amino acid residues having side chains with chemical similarity have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A conservative substitution may also include the use of a chemically derivatized residue in place of a non-derivatized residue. A chemical derivative residue is a residue chemically derivatized by reaction of a functional group of the residue. Examples of such chemical derivatives include, but are not limited to, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters, or other types of esters or hydrazides. Free hydroxyl groups may be derivatized to form O-acyl or O-alkyl derivatives. Also included as chemical derivatives are those polypeptides which contain one or more naturally-occurring amino acid derivatives of the twenty standard amino acids. For example, 4-hydroxyproline may be substituted for proline; 5-hydroxylsine may be substituted for histidine; homoserine may be substituted for serine; and ornithine may be substituted for lysine.

An amino acid residue of the polypeptide can be replaced by another amino acid residue in a non-conservative substitution. In some cases, a non-conservative substitution will not alter the relevant properties of the polypeptide. The relevant properties can be, without limitation, ability to bind to an antibody that recognizes the marker.

Frequently, two sequences will be capable of hybridizing when their sequences are less 100% complementary. For example, when allowed to interact under stringent conditions, two sequences with at least 85% sequence identity, at least 90% sequence identity, or 95% or greater sequence identity, can hybridize. One example of stringent conditions is a buffer including 0.5 M sodium phosphate (pH 7.2), 7% (w/v) SDS, and 1 mM EDTA, at 65° C. Methods for selecting and preparing a nucleotide capable of hybridizing to a desired sequence are known.

The ligand can be an antibody or a receptor. For example, the ligand can be a formyl peptide receptor or fragment thereof, a formyl peptide receptor sub-unit or fragment thereof, or an encoded sequence of binding region of a formyl peptide receptor. See, for example, residues 84 to 100 of the sequence of the human formyl peptide receptor described in Lala, A., et al., Human formyl peptide receptor function role of conserved and nonconserved charged residues Eur. J. Biochem. 254, 495-499, which is incorporated by reference in its entirety. The ligand can be a combination of these. Ligands such as these can detect any mitochondrial marker released, and can detect the mitochondrial marker with more specificity.

An antibody is an immunoglobulin molecule or an immunologically active portion of an immunoglobulin molecule, i.e., a molecule that contains an antigen binding site that specifically binds an antigen. The immunoglobulin molecules can be of any class (e.g., IgG, IgE, IgM, IgD and IgA) or subclass of immunoglobulin molecule. Antibodies includes, but are not limited to, polyclonal, monoclonal, bispecific, humanised and chimeric antibodies, single chain antibodies, Fab fragments and F(ab′)2 fragments, fragments produced by a Fab expression library, anti idiotypic (anti Id) antibodies, and epitope binding fragments of any of the above. An antibody, or generally any molecule, binds specifically to or has affinity for an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions.

Antibodies prepared against a peptide may be tested for activity against that peptide as well as the full length protein. Antibodies may have affinities of at least about 10⁻⁶ M, 10⁻⁷ M, 10⁻⁸ M, 10⁻⁹ M, 10⁻¹⁰ M, 10⁻¹¹ M or 10⁻¹² M toward the peptide and/or the full length polypeptide.

In general, polyclonal antibodies that recognize a particular polypeptide can be generated by immunizing a mammal (such as a mouse or rabbit) with the polypeptide. The polypeptide can be a mitochondrial marker, a fragment or cleavage product of a mitochondrial marker, or a a mitochondrial marker analog. The polypeptide can include other sequences besides a mitochondrial marker sequence. For example, the a mitochondrial marker analog can be biologically active (for example, sharing some or all of the biological effects of the a mitochondrial marker) or biologically inactive. Whether biologically active or inactive, the analog can serve as an antigen for generating antibodies that recognize, for example, a mitochondrial marker. The polypeptide antigen can be linked to a larger polypeptide by chemical methods, or cloned and expressed as a fusion with a larger polypeptide. The polypeptide antigen can be injected as a mixture with an adjuvant, such as Freund's complete adjuvant. An ELISA assay can be used to determine the titer of antibodies in serum collected from the animal. Detailed procedures for the generation of polyclonal antibodies can be found, for example, in Current Protocols in Immunology, 2001, John E. Coligan, ed., John Wiley & Sons.

In general, monoclonal antibodies that recognize a particular polypeptide can be generated by immunizing a BALB/c mouse with the polypeptide. The polypeptide can be linked to a larger polypeptide by chemical methods, or cloned and expressed as a fusion with a larger polypeptide. The polypeptide antigen can be injected as a mixture with an adjuvant, such as Freund's complete adjuvant. Spleen cells from the immunized mouse can be fused with myeloma cells to form immortal, antibody-expressing cells. Cells that express an antibody having specificity for the desired polypeptide can be isolated and used to produce additional quantities of the monoclonal antibody. Detailed procedures for the generation of monoclonal antibodies can be found, for example, in Current Protocols in Immunology, 2001, John E. Coligan, ed., John Wiley & Sons.

When an antibody is made by the methods described above, it can be described as being derived from a mammal (for example, a mouse or rabbit in the description above). A monoclonal antibody produced from a hybridoma cell culture is considered to be derived from the mammal, since the hybridoma is made by fusing cells from the mammal immunized with an antigen.

The level of a mitochondrial marker in a sample can be measured qualitatively or quantitatively using an assay, for example, in an immunochromatographic format. A qualitative assay can be distinguish between the presence or absence of a marker, or can distinguish between categories of marker levels in a sample, such as absent, low concentration, medium concentration or high concentration, or combinations thereof. A quantitative assay can provide a numerical measure of a marker in a sample. The assay can include contacting a marker with an antibody that recognizes that particular marker, detecting the marker by mass spectrometry, assaying a sample including cells for expression (e.g., of mRNA or polypeptide) of the marker gene by the cells, or a combination of measurements. For example, the assay can include contacting a sample with an antibody that recognizes the marker and a mass spectrometry measurement.

The a mitochondrial marker can be detected in blood, serum, plasma or other bodily fluids which can be obtained from a body, such as interstitial fluid, urine, whole blood, saliva, serum, lymph, gastric juices, bile, sweat, tear fluid and brain and spinal fluids. Bodily fluids may be processed (e.g., serum) or unprocessed.

Marker levels may be provided in units of concentration, mass, moles, volume or any other measure indicating the amount of marker present.

The respective levels of the a mitochondrial marker and secondary markers may be measured using an immunoassay, for example, by contacting the sample with an antibody that binds specifically to the marker and measuring any binding that has occurred between the antibody and at least one species in the sample. Such assays may be competitive or non-competitive immunoassays. The assay can be homogeneous or heterogeneous. The analyte to be detected can be caused to bind with a specific binding partner such as an antibody which has been labelled with a detectable species such as a latex or gold particle, a fluorescent moiety, an enzyme, an electrochemically active species, etc. Alternatively, the analyte can be labelled with any of the above detectable species and competed with limiting amounts of specific antibody. The presence or amount of analyte present is then determined by detection of the presence or concentration of the label. Such assays may be carried out in the conventional way using a laboratory analyser or with point of care or home testing device, such as the lateral flow immunoassay as described in EP291194, which is incorporated by reference in its entirety.

In one embodiment, an immunoassay is performed by contacting a sample from a subject to be tested with an appropriate antibody under conditions such that immunospecific binding can occur if the marker is present, and detecting or measuring the amount of any immunospecific binding by the antibody. The antibody may be contacted with the sample for at least about 10 minutes, 30 minutes, 1 hour, 3 hours, 5 hours, 7 hours, 10 hours, 15 hours, or 1 day. Any suitable immunoassay can be used, including, without limitation, competitive and non competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays and protein A immunoassays.

In another embodiment, antibodies to a mitochondrial marker can be immobilized on a surface in a sandwich assay. A sample of interest is allowed to interact with the immobilized antibodies. If a mitochondrial marker is present in the sample, it will be bound by the antibodies and thus become immobilized. After incubation, the surface can be washed prior to addition of a second antibody to a mitochondrial marker. The second antibody can recognize a different epitope of a mitochondrial marker than the immobilized antibody. The second antibody may recognize a mitochondrial marker.

If a mitochondrial marker was present in the initial sample, an immobilized antibody/a mitochondrial marker/second antibody sandwich forms. The second antibody can be coupled to a colored material, or alternatively, the sandwich can then be detected by a third antibody. Typically the third antibody is an anti-IgG antibody derived from a different species than the second antibody. For example, if the second antibody to a mitochondrial marker related peptide is a mouse IgG, then the third antibody can be a goat anti-mouse IgG antibody or a rabbit anti-mouse IgG antibody.

The second or third antibody can produce a detectable change when bound to its target. For ease of detection of the sandwich, the second or third antibody can be associated with a color-developing reagent. The color-developing reagent can be a colored material (such as a dye or colored latex particle) or a reagent capable of converting a colorless material to a colored material. One such reagent is a peroxidase enzyme linked to the third antibody. In the presence of appropriate substrates, the peroxidase enzyme can produce a colored product, which is easily detected by virtue of its color. The use of an enzyme (or other catalyst) to produce a detectable change in samples having a mitochondrial marker can increase the sensitivity of the assay. Other methods of detecting an antigen (such as a mitochondrial marker) using antibodies to the antigen are known. Examples of immunochromatographic tests and test result readers can be found in, for example, U.S. Pat. Nos. 5,504,013; 5,622,871; 6,235,241; and 6,399,398, each of which is incorporated by reference in its entirety.

In cardiac cells, mitochondria exist in two functionally distinct populations. Subsarcolemmal mitochondria are located beneath the plasma membrane, whereas interfibrillar mitochondria are present between the myofibrils; intracellular arrangement and regulation of mitochondrial respiration are tissue specific—in cardiac muscle, mitochondria are localized in the intermyofibrillar space at the level of the A-band of sarcomeres. See, for example, Palmer, J W, Tandler B, and Hoppel C L. Biochemical properties of subsarcolemmal and interfibrillar mitochondria isolated from rat cardiac muscle. J. Biol. Chem. 252: 8731-8739, 1977; and Boudina, S. et al., Alteration of mitochondrial function in a model of chronic ischemia in vivo in rat heart. Am. J. Physiol. Heart Circ. Physiol. 2002 March; 282(3):H821-31, each of which is incorporated by reference in its entirety.

In cardiac cells, mitochondria are located in functional complexes with sarcomeres and sarcoplasmic reticulum to achieve the most effective regulation of cellular energetics. These complexes, or intracellular energetic units (ICEUs) represent the basic pattern of organization of energy metabolism in cardiac and oxidative muscle cells. Mitochondria are arranged in a highly ordered crystal-like pattern in a muscle-specific manner. Structural connections between mitochondria and sarcomeres inside ICEUs are so strong that there exists a direct link between sarcomere length and regulation of mitochondrial function. Organization of mitochondria into ICEUs results in the heterogeneity of the intracellular diffusion of ADP (and ATP), a phenomenon which is in agreement with the general theories of the compartmentation of adenine nucleotides in the cardiac cells (see, for example, Vendelin, M., et al., Mitochondrial regular arrangement in muscle cells: a “crystal-like” pattern, Am. J. Physiol. Cell Physiol. 2005 March; 288(3):C757-67; and Saks, V. A., et al., Intracellular energetic units in red muscle cells, Biochem. J. 2001, 356, 643-657, each of which is incorporated by reference in its entirety).

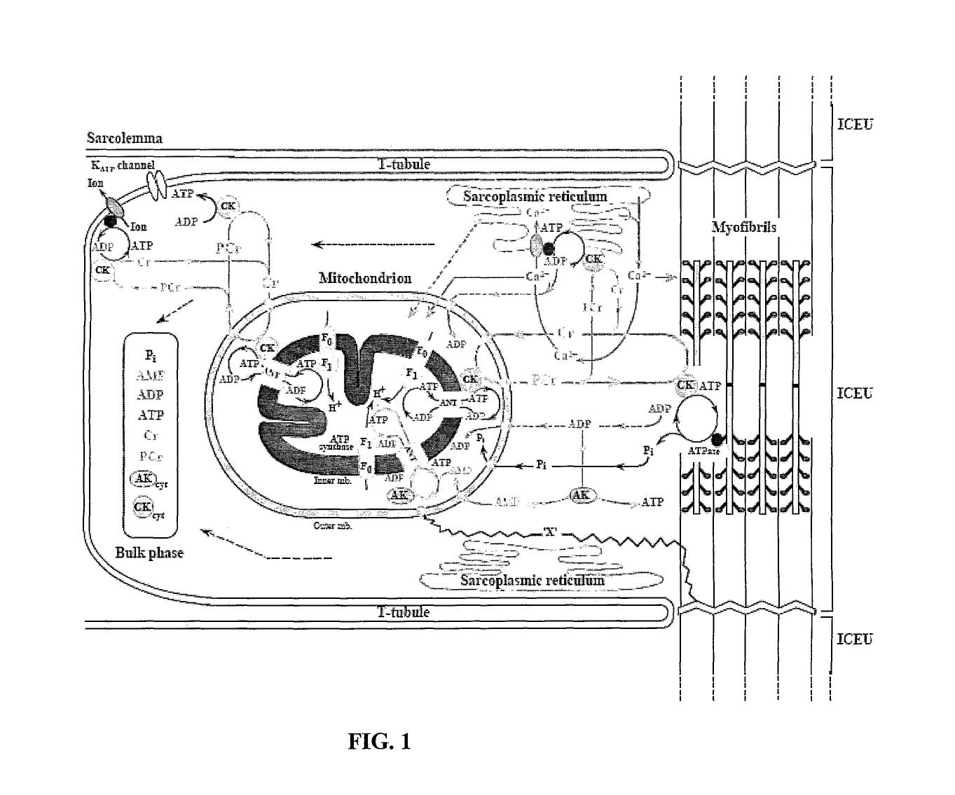

In the normal cardiomyocyte, efficient energy transfer between cytosol and mitochondria depends on two organizational aspects of the mitochondrial isoenzyme of creatine kinase, which catalyses the forward reaction: Creatine+ATP->phosphocreatine+ADP. FIG. 1 schematically illustrates metabolic compartmentalization in cardiac cells. Functional coupling and compartmentation both depend strongly on the structure-function of the intermembrane space. Mitochondria in ischemic zones are dramatically changed with detachment of mitochondria from myofibrils leading to destruction of function. These alterations result in the impairment of intracellular energy transfer (channeling) from mitochondria to ATP-utilizing sites (see, e.g., Boudina, S. et al., Am. J. Physiol. Heart Circ. Physiol. 2002 March; 282(3):H821-31).

Energy production in the heart is mainly supported by mitochondrial function. Investigations have focused on mitochondrial alterations and energy production during acute ischemia and reperfusion in vitro. For example, ischemia followed by reperfusion is known to negatively affect mitochondrial function, by inducing a deleterious condition called mitochondrial permeability transition (MPT). The MPT is responsible for mitochondrial dysfunction and can ultimately lead to cell death. N-formylmethionine containing peptides are released from degenerating mitochondria at sites of tissue damage and this might play a role in the accumulation of inflammatory cells observed at these sites. It is plausible that the N-formylmethionine peptide Nourin-1 is derived from mitochondrial degradation. See, for example, Kay L, et al., Alteration in the control of mitochondrial respiration by outer mitochondrial membrane and creatine during heart preservation. Cardiovasc Res. 34: 547-556, 1997; Kay L, Rossi A, and Saks V. Detection of early ischemic damage by analysis of mitochondrial function in skinned fibers. Mol. Cell. Biochem. 174: 79-85, 1997; Kay L, Saks V A, and Rossi A. Early alteration of the control of mitochondrial function in myocardial ischemia. A Mol. Cell. Cardiol. 29: 3399-3411, 1997; Carp H., J. Exp. Med. 1982 Jan. 1; 155(1):264-75, and Mair, J. Clin Chem Lab Med. 37(11/12):1077-1084, 1999, each of which is incorporated by reference in its entirety.

Mitochondria are unique among organdies of animal cells in that they contain their own DNA (mitochondrial DNA, or mtDNA). Of the 37 genes that coded by mitochondrial DNA, 13 are translated into proteins, all of which are localized to the inner-mitochondrial membrane as components of the respiratory chain complexes (i.e., complexes I, II, III, IV and V; see, for example, The human mitochondrial proteome: oxidative stress, protein modifications and oxidative phosphorylation Int. J. Biochem. Cell. Biol. 2005 May; 37(5):927-34, which is incorporated by reference in its entirety). The respiratory chain includes complex I (NADH:ubiquinone oxidoreductase), complex II (succinate: ubiquinone oxidoreductase), complex III (ubiquinol:cytochrome c oxidoreductase), and complex IV (cytochrome c oxidase), which function together to generate an electrochemical potential across the inner mitochondrial membrane. Complex V (F₁F₀-ATP synthase) uses this electrochemical proton gradient to synthesize ATP. These complexes have been extensively studied, successfully purified and characterized, both at the proteomic and genomic level. See, for example, The mitochondrial electron transport and oxidative phosphorylation system Annu. Rev. Biochem. 1985; 54: 1015-69; and Assembly of respiratory complexes I, III, and IV into NADH oxidase supercomplex stabilizes complex I in Paracoccus denitrifcans. J. Biol. Chem. 2004 Feb. 6; 279(6):5000-7, each of which is incorporated by reference in its entirety.

Because mitochondria are present in animal cells, mitochondrial components (e.g., a mitochondrial marker) can be detected in a sample taken from a subject animal, such as, for example, a human subject, or a non-human subject such as, for example, a bird, a mouse, a rat, a rabbit, a pig, a sheep, a goat, a cow, or another mammal.

The mitochondrial respiratory chain is a major source of reactive oxygen species (ROS) under pathological conditions including myocardial ischemia and reperfusion. Limitation of electron transport by the inhibitor rotenone immediately before ischemia decreases the production of ROS in cardiac myocytes and reduces damage to mitochondria. ROS are produced from mitochondrial complex I by the NADH dehydrogenase located in the matrix side of the inner membrane and are dissipated in mitochondria by matrix antioxidant defenses. See, for example, Chen Q, et al. Production of reactive oxygen species by mitochondria: central role of complex III. J. Biol. Chem. 2003 Sep. 19; 278(38):36027-31, which is incorporated by reference in its entirety. ROS contribute to a number of pathological processes including aging, apoptosis, and cellular injury during ischemia and reperfusion. The mitochondrial electron-transport chain is the main source of ROS during normal metabolism. The rate of ROS production from mitochondria is increased in a variety of pathologic conditions including hypoxia, ischemia, and reperfusion. Most of the ROS radicals are produced at Complex I, and high rates of production of ROS are features of respiratory chain-inhibited mitochondria and of reversed electron flow arising in conditions of ischemia (see, e.g., Kudin A P, et al. Characterization of superoxide-producing sites in isolated brain mitochondria. J. Biol. Chem. 2004 Feb. 6; 279(6):4127-35, which is incorporated by reference in its entirety).

Complex I is the entry point for electrons into the respiratory chains of many bacteria and mitochondria of most eukaryotes. It couples electron transfer with the translocation of protons across the membrane, thus providing the proton motive force essential for energy-consuming processes. Following two-dimensional SDS-PAGE and electroblotting, a mixture of specific antibodies was used to identify the location of assembled complexes and dissociated subunits purified from mitochondria. Antibodies identified supercomplexes a, b, and c and individual complexes III and IV, but intact individual Complex I was not present. Complex I is stabilized by super-assembly into the NADH oxidase complex and appears to easily dissociate. See, for example, J. Biol. Chem. 2004 Feb. 6; 279(6):5000-7, which is incorporated by reference in its entirety.

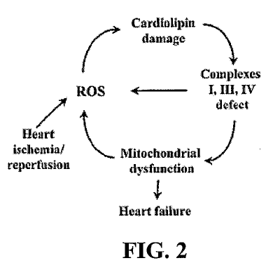

The activity of complex I is reduced in mitochondria isolated from ischemic and reperfused rat heart. The mitochondrial content of cardiolipin, which is required for optimal activity of complex I, decreases as function of ischemia and reperfusion. Cardiolipin is recognized as a relatively early target of ischemic mitochondrial damage. The simple model illustrated in FIG. 2 summarizes the link between ROS, mitochondrial complex defects, and mitochondrial dysfunction. See, for example, Paradies G, et al., Decrease in mitochondrial complex I activity in ischemic/reperfused rat heart: involvement of reactive oxygen species and cardiolipin. Circ. Res. 2004 Jan. 9; 94(1):53-9, which is incorporated by reference in its entirety.

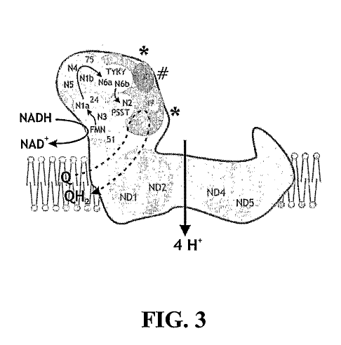

Mitochondrial complex I catalyzes electron transfer from NADH to ubiquinone in a process coupled to proton transport across the inner mitochondrial membrane. Complex I is made of more than 30 subunits, the majority of which are encoded by nuclear genes and imported from the cytoplasm. However, seven subunits are coded for by mitochondrial genes (ND1, -2, -3, -4, -4L, -5, and -6; see, e.g., Remade C, et al. Mutants of Chlamydomonas reinhardtii deficient in mitochondrial complex I: characterization of two mutations affecting the nd1 coding sequence. Genetics 2001 July; 158(3):1051-60, which is incorporated by reference in its entirety). An arrangement of the subunits is shown in FIG. 3; Table 1 summarizes the nomenclature for the subunits found in mammals, bacteria, and yeast.

TABLE 1
Complex I subunit symbol

| Bovine | Y. lipolytica | E. coli |
|---|---|---|
| 75 kDa | NUAM | NuoG |
| 51 kDa | NUBM | NuoF |
| 49 kDa | NUCM | NuoD |
| 30 kDa | NUGM | NuoC |
| 24 kDa | NUHM | NuoE |
| TYKY | NUIM | NuoI |
| PSST | NUKM | NuoB |
| ND1 | ND1 | NuoH |
| ND2 | ND2 | NuoN |
| ND3 | ND3 | NuoA |
| ND4 | ND4 | NuoM |
| ND4L | ND4L | NuoK |
| ND5 | ND5 | NuoL |
| ND6 | ND6 | NuoJ |

Release of mitochondrial proteins has been proposed as a sensitive indicator of cellular damage that might result in mitochondrial proteins into the circulation. Importantly, proteomic analysis of ischemic hearts revealed profound changes in enzymes related to energy metabolism, e.g., NADH dehydrogenase and ATP synthase, with partial fragmentation of these mitochondrial enzymes. An amino acid sequence of the NADH dehydrogenase subunit 1 (ND1) was found to exhibit FPR-binding properties. See, for example, Shawar S M, et al. Peptides from the amino-terminus of mouse mitochondrially encoded NADH dehydrogenase subunit 1 are potent chemoattractants. Biochem. Biophys. Res. Commun. 1995 Jun. 26; 211(3):812-8; and Mayr M, et al. Ischemic preconditioning exaggerates cardiac damage in PKC-delta null mice. Am. J. Physiol. Heart Circ. Physiol. 2004 August; 287(2):H946-56, each of which is incorporated by reference in its entirety.

The N-terminus of ND1 was found to have significant chemotactic activity (see, e.g., Shawar S M, et al. Biochem. Biophys. Res. Commun. 1995 Jun. 26; 211(3):812-8). The ND1 peptide is believed to interact with FPRL1 and not FPR (see, e.g., Chiang N, et al. J. Exp. Med. 2000; 191:1197-207). The N-terminus features an N-terminal methionine followed by two hydrophobic amino acids. Note that Freer suggested that the ligand for FPR occupies a hydrophobic pocket in the receptor (Freer, R. J., et al. (1982) Formyl peptide chemoattractants: a model of the receptor on rabbit neutrophils. Biochemistry 21, 257-263, which is incorporated by reference in its entirety).

!Peptide? Sequence

| | |
|---|---|
| fMLP | fMLF |
| ND1α₁₋₁₂ | MFFINILTLLVP |

Through reductive evolution, the complement of genes constituting the original eubacterial predecessors of modern-day mitochondria have been either lost or transferred from mitochondrial DNA to the nuclear genome (see, for example, Andersson, S. G (1998). The genome sequence of Rickettsia prowazekii and the origin of mitochondria. Nature 396, 133-140, which is incorporated by reference in its entirety). The mitochondrion has also acquired new proteins and functionality. A systematic survey of mitochondrial proteins from brain, heart, kidney, and liver of C57BL6/J mice was performed by Mootha. Mitochondrial proteins from each tissue were solubilized and size separated by gel filtration into a batch of approximately 15-20 fractions. These proteins were then digested and analyzed by liquid chromatography; the proteins varied in molecular weight and isoelectric point. An important finding was a high percentage of hydrophobic and membrane proteins that had up to now been seriously underrepresented by 2-DE gel protocols. See, for example, Integrated Analysis of Protein Composition, Tissue Diversity, and Gene Regulation in Mouse Mitochondria Cell, Vol. 115, 629-640, Nov. 26, 2003; and The human mitochondrial proteome: oxidative stress, protein modifications and oxidative phosphorylation The International Journal of Biochemistry & Cell Biology 37 (2005) 927-93, each which is incorporated by reference in its entirety.

A mitochondrial protein sequence database (MitoProteome) was generated from experimental evidence and public databases, and containing both mitochondrial- and nuclear-encoded entries. The initial release contains 847 human mitochondrial proteins, 615 of which were experimentally determined by mass spectrometry. See, e.g., Steven W. Taylor, et al. Characterization of the human heart mitochondrial proteome. Nature Biotechnology 2003, 21, 3 pp 281-286, which is incorporated by reference in its entirety. Less than 5% of the encoded proteins have a molecular weight of less than 10 kDa.

For example, two of the low molecular weight mitochondrial proteins identified from human mitochondria are:
NADH dehydrogenase (ubiquinone) 1 beta subcomplex, 1, 7 kDa;
NADH dehydrogenase (ubiquinone) 1 beta subcomplex, 1 (7 kD, MNLL) (Homo sapiens), sequence:

MICWRHPSAPCGRGEWQVPRSQLPLARVEFPVALGLGVAVGAEAAAIMVN
LLQIVRDHWVHVLVPMGFVIGCYLDRKSDERLTAFRNKSMLFKRELQPSE
EVTWK

Cytochrome c oxidase subunit VIIc precursor; cytochrome-c oxidase chain VIIc (Homo sapiens), sequence:

MLGQSIRRFTTSVVRRSHYEEGPGKNLPFSVENKWSLLAKMCLYFGSAFA
TPFLVVRHQLLKT

The chemokines are 8-14 kda-secreted cytokines, and four subfamilies have been discovered including: CXC(a), CC(b), C(g) and CX3C. Haddad has summarized currently known cytokines and their receptors (see Table 2 below, and Murphy P M, et al. (2000). International union of pharmacology. XXII. Nomenclature for chemokine receptors. Pharmacol Rev. 52: 145-176; and Cytokines and related receptor-mediated signaling pathways Biochem. Biophys. Res. Commun. 2002 Oct. 4; 297(4):700-13, each of which is incorporated by reference in its entirety). Most cytokines are unrelated in terms of sequence, although some can be grouped into families or are classified into categories according to the types of secondary and tertiary structure. IFN-α, IFN-β, IFN-X, IL-2, IL-3, IL-4, IL-5, IL-6, IL-7, IL-9, G-CSF, M-CSF, GM-CSF, and PDGF, for example, have α-helical secondary structure. Beta-structural cytokines include IL-1a, IL-1b, TNF-α, TNF-β, and FGF. Composite structures (α and β) are observed with IL-8, IFN-α, IP-10, PF-4, GRO, and 9E3. While none of these cytokines have molecular weights of <3 kDa, data published results on Lkn-1, CKb8 and other CC chemokines (i.e., HCC1, MCP-1, MCP-2, MIP-1b), suggest that the processing of the N-terminus of some members of b-chemokines, including CKb8-1, may represent a novel mechanism to increase the diversity of inflammatory effects inherent to these ligands. See, for example, Elagoz A, et al. A truncated form of CKbeta8-1 is a potent agonist for human formyl peptide-receptor-like 1 receptor. Br. J. Pharmacol. 2004 January; 141(1):37-46, which is incorporated by reference in its entirety. It is possible that Nourin-1 is a ligand released from a high molecular weight species.

TABLE 2
Structural families of cytokines and cytokine receptors

| Cytokine family | Members | Receptor type |
|---|---|---|
| Haematopoietins | IL-2, IL-3, IL-4, IL-5, IL-6, IL-7, IL-9, | Cytokine receptor class I |
| (four α-helical bundles) | IL-13, G-CSF, GM-CSF, CNTF, OSM, LIF, and EPO | |
| | IL-10, IFN-α, IFN-β, and IFN-γ | Cytokine receptor class II |
| | M-CSF | Tyrosine kinase |
| EGF (β-sheet) | EGF and TGF-α | Tyrosine kinase |
| β-Trefoil | FGF-α and FGF-β | Split tyrosine kinase |
| | IL-1α, IL-1β, and IL-1ra | IL-1 receptor |
| TNF (Jelly roll motif) | TNF-α, TNF-β, LT-β | NGF/TNF receptor |
| Cysteine knot | NGF | NGF/TNF receptor |
| | TGF-β₁, TGF-β₂, TGF-β₃ | Scrine/threonine kinase |
| | PDGF and VEGF | Tyrosine kinase |
| Chemokines (triple-stranded, | IL-8, MIP-1α, MIP-1β, MIP-2, PF-4, PBP, | Rhodopsin superfamily |
| anti-parallel β-sheet | I-309/TCA-3, MCP-1, MCP-2, MCP-3, γIP-10 | |
| in Greek key motif) | | |

Ligands to FPR can be distinguished from ligands that bind to two related receptors, referred to as FPR-like 1 (FPRL1) and FPR-like 2 (FPRL2). These receptors, unlike FPR, are low-affinity receptors for the agonist formyl-Met-Leu-Phe (fMLP) and are only activated by high (micromolar) concentrations. See, for example, Gao, J. L., and P. M. Murphy. 1993. Species and subtype variants of the N-formyl peptide chemotactic receptor reveal multiple important functional domains. J. Biol. Chem. 268:25395; and Lavigne M C, Murphy P M, Leto T L, Gao J L. The N-formylpeptide receptor (FPR) and a second G(i)-coupled receptor mediate fMet-Leu-Phe-stimulated activation of NADPH oxidase in murine neutrophils. Cell Immunol. 2002 July-August; 218(1-2):7-12, each of which is incorporated by reference in its entirety.

Known ligand for FPR include several novel host-derived FPR ligands which are not formylated and do not show homology in their amino acid sequences. See, for example, Walther A, Riehemann K, Gerke V. A novel ligand of the formyl peptide receptor: annexin I regulates neutrophil extravasation by interacting with the FPR. Mol. Cell 2000; 5:831-40; Le Y, Murphy P M and Wang J M (2002) Formyl-peptide receptors revisited. Trends Immunol. 23:541-48; Murphy P M (1996) The N-formyl peptide chemotactic receptors, in “Chemoattractant ligands and their receptors” (Horuk R ed) pp 269, CRC Press, Inc., Boca Raton; and Prossnitz E R and Ye R D (1997) The N-formyl peptide receptor: a model for the study of chemoattractant receptor structure and function. Pharmacol. Ther. 74:73-102; each of which is incorporated by reference in its entirety. The only FPR agonist yet identified, Annexin I and its N-terminal peptides (AA1-26 and AA9-25), were verified using the fMLP antagonists (Boc-Met-Leu-Phe; Boc1, and Boc-Phe-Leu-Phe-Leu-Phe; Boc2) on the anti-migratory activity of the annexin I peptides Ac1-26 and Ac9-25 (see Walther, A. et al. (2000) Mol. Cell. 5, 831-840).

Known ligands for FPR, FPRL1, and FPRL2 are listed in Table 3 (see, for example, Partida-Sanchez, S. Chemotaxis and calcium responses of phagocytes to formyl peptide receptor ligands is differentially regulated by cyclic ADP ribose, J. Immunol. 2004 Feb. 1; 172(3):1896-906, which is incorporated by reference in its entirety).

TABLE 3
Host derived agonists

| Agonist | Derived from | target |
|---|---|---|
| MHC binding peptide | NADH dehydrogenase | FPRL1 |
| | subunit I | |
| LL-37 | hCAP18 (aa1-37) | FPRL1 |
| Ac1-26 | annexin1 (aa1-26) | FPR |
| Ac9-25 | annexin1 (aa9-25) | FPR |
| D2D3₈₈₋₂₇₄ | uPAR (aa88-274) | FPRL1 |
| LXA4 | lipid metabolite | FPRL1, mLXA4R |
| SAA | acute phase protein | FPRL1, mFPR2 |
| Aβ₄₂ | APP (aa1-42) | FPRL1, mFPR2 |
| PrP106-126 | Prion (aa106-126) | FPRL1 |

A peptide fragment of NADH dehydrogenase subunit 1 having the sequence MYFINILTL, is specific for FPRL1. See, for example, Chiang N, Fierro I M, Gronert K, Serhan C N. Activation of lipoxin A4 receptors by aspirin-triggered lipoxins and select peptides evokes ligand-specific responses in inflammation. J. Exp. Med. 2000; 191:1197-20, which is incorporated by reference in its entirety.) LL-37 is chemotactic for, and can induce calcium mobilization in, human monocytes and formyl peptide receptor-like 1 (FPRL1)-transfected human embryonic kidney 293 cells. (see De Yang, LL-37, the Neutrophil Granule- and Epithelial cell-derived Cathelicidin, Utilizes Formyl Peptide Receptor-like 1 (FPRL1) as a Receptor to Chemoattract Human Peripheral Blood Neutrophils, Monocytes, and T Cells. J. Exp. Med. 192, 7, 1069-1074, which is incorporated by reference in its entirety).

FPRL1 binds serum amyloid A (SAA), Beta amyloid peptide, prion protein peptide, and the lipid metabolite lipoxin A. See, for example, Su, S B, et al., Activation of a Chemoattractant Receptor FPRL1 by SAA, J. Exp. Med. 189, 2, 395-402; and Partida-Sanchez, S., J. Immunol. 2004 Feb. 1; 172(3):1896-906, each of which is incorporated by reference in its entirety. A novel role for FPRL1 as a high-affinity b-chemokine receptor for an N-terminally truncated form of the CKb8 (also known as CCL23/MPIF-1) splice variant CKb8-1 (22-137 aa) has been described (see, for example, Elagoz A, et al., A truncated form of CKbeta8-1 is a potent agonist for human formyl peptide-receptor-like 1 receptor. Br J Pharmacol. 2004 January; 141(1):37-46, which is incorporated by reference in its entirety).

FPRL2 is expressed in monocytes but not in neutrophils, and is not activated by N-formylpeptides (see, e.g., Elagoz A, et al., Br J Pharmacol. 2004 January; 141(1):37-46, which is incorporated by reference in its entirety). Thus far, no ligands have been identified for FPRL2.

Further evidence a putative ligand interacts with the FPR receptor might be provided from the observation that the receptors, FPR and FPRL1, can be distinguished by their reliance on cyclic ADP ribose (cADPR) for calcium signaling (Partida-Sanchez, S., J. Immunol. 2004 Feb. 1; 172(3):1896-906); this knowledge should provide an experimental method to demonstrate FPR specificity of the putative ligand.

The first extracellular loop and its adjacent transmembrane domains of FPR are essential for high affinity binding of fMLP. Information on the sequence and binding site structure of FPR is available. See, for example, Miettinen, H. M., et al. The ligand binding site of the formyl peptide receptor maps in the transmembrane region. J. Immunol. 1997 159:4045-4054; and Lala, A., et al., Human formyl peptide receptor function role of conserved and nonconserved charged residues Eur. J. Biochem. 254, 495-499, each of which is incorporated by reference in its entirety.

The human FPR exists in several isoforms (FPR-26, FPR-98 and FPR-G6). It has a molecular weight of 68 kDa. The FMLP receptor on human neutrophils has been reported to consist of multiple components, the major species being a glycoprotein of 55,000-70,000 Da. See, for example, Seifert R, and Wenzel-Seifert K, The human formyl peptide receptor as model system for constitutively active G-protein-coupled receptors. Life Sci. 2003 Sep. 19; 73(18):2263-80; Goetzl E J, (1981) Biochemistry, 20, 5717; Quehenberger O, Prossnitz E R, Cochrane C G and Ye R D, Absence of G proteins in the Sf9 insect cell. Characterization of the uncoupled recombinant N-formyl peptide receptor. J. Biol. Chem. 267: 19757-19760, 1992; and De Nardin E, Radel S J and Genco R J, Isolation and partial characterization of the formyl peptide receptor components on human neutrophils. Biochem. Biophys. Res. Commun. 174: 84-89, 1991, each of which is incorporated by reference in its entirety.

Deglycosylation with endoglycosidase F leaves a core peptide of ˜33,000 Da, which is still able to bind the ligand. Isolation of a cDNA that codes for the human N-formylpeptide receptor has been reported. Using peptide analogs to different domains of the receptor, Radel et al. have shown that charged residues in the first extracellular loop play a critical role in ligand binding. See, for example, Lala, A., et al., Recombinant expression and partial characterization of the human formyl peptide receptor. Biochim. Biophys. Acta 1993, 1178, 302-306; Malech H L, et al., Asparagine-linked oligosaccharides on formyl peptide chemotactic receptors of human phagocytic cells. J. Biol. Chem. 260: 2509-2514, 1985; Boulay F, et al., Synthesis and use of a novel N-formyl peptide derivative to isolate a human N-formyl peptide receptor cDNA. Biochem Biophys. Res. Commun. 168: 1103-1109, 1990; Radel S J, et al. Localization of Liand-binding regions of human formyl peptide receptor. Biochem. Int. 25: 745-753, 1991; and Radel S J, et al., Structural and functional characterization of the human formyl peptide receptor ligand-binding region. Infect. Immunol. 62: 1726-1732, 1994, each of which is incorporated by reference in its entirety.

Recombinant FPR was prepared by expression in E. coli followed by purification using gel filtration and affinity chromatography using an fMLP-Sepharose column and elution with fMLP resulting in approximately 1 mg yield and the recombinant FPR retained ligand binding capacity. Initial studies on the FPR ligand binding domains suggested that the ligand might occupy a hydrophobic pocket in the receptor. A synthetic 17-aa peptide (RKAMGGHWPFGWFLCKF), corresponding to residues 84 to 100 in the first extracellular domain of the FMLP receptor, was the strongest inhibitor of ligand binding to the 68-kDa protein. See, for example, Lala A, Sojar H T, De Nardin E, Expression and Purification of Recombinant Human N-Formyl-L-leucyl+phenylalanine (FMLP) Receptor, Biochemical Pharmacology, Vol. 54, pp. 381-390, 1997; Lala, A. & De Nardin, E. (1996) Role of Asp in ligand binding of human FMLP receptor. J. Dent. Res. 75, (Abstract 3204); Freer, R. J., et al. (1982) Formyl peptide chemoattractants: a model of the receptor on rabbit neutrophils. Biochemistry 21, 257-263; and Lala, A., et al., Biochim. Biophys. Acta 1993, 1178, 302-306, each of which is incorporated by reference in its entirety.

Functional studies of formyl peptide receptors have been performed by using neutrophils and monocytes, the expression of these receptors have been demonstrated in other cell types. For instance, hepatocytes, immature dendritic cells, astrocytes, microglial cells, and the tunica media of coronary arteries express the high-affinity FPR. While the chemoattractant activity has been demonstrated using a neutrophil model, it is important to recognize that FPR receptors are present elsewhere. Importantly, related to ACS, endogenous formyl peptides are released by eukaryotic mitochondria from necrotic cells and induce chemotaxis using FPR expressed by thrombin-activated platelets. See, e.g., McCoy R, et al. N-formylpeptide and complement C5a receptors are expressed in liver cells and mediate hepatic acute phase gene regulation. J. Exp. Med. 1995; 182:207-17; Sozzani S, et al. Migration of dendritic cells in response to formyl peptides C5a and a distinct set of chemokines. J. Immunol. 1995; 155:3292-5; Lacy M, et al. Expression of the receptors for the C5a anaphylatoxin, interleukin-8 and FMLP by human astrocytes and microglia. J. Neuroimmunol. 1995; 61:71-8; Keitoku M, et al. FMLP actions and its binding sites in isolated human coronary arteries. J. Mol. Cell. Cardiol. 1997; 29:881-94; and Czapiga M et al., Human platelets exhibit chemotaxis using functional N-formyl peptide receptors. Exp. Hematol. 2005 January; 33(1):73-84, each of which is incorporated by reference in its entirety.

N-formylmethionine peptides can be derived from invading bacteria, suggesting that a formylmethionine peptide present in the low molecular weight sample might be of bacterial origin (i.e., a contaminant). However, mitochondria are known to initiate protein synthesis with an N-formylmethionine residue, and preparations of disrupted human mitochondria or mitochondrial proteins cause neutrophil accumulation (see, for example, Carp H. Mitochondrial N-formylmethionyl proteins as chemoattractants for neutrophils. J. Exp. Med. 1982 Jan. 1; 155(1):264-75, which is incorporated by reference in its entirety). Mitochondria are usually considered to be the powerhouse of the cell and to be responsible for the aerobic production of ATP. However, many eukaryotic organisms are known to possess anaerobically functioning mitochondria, which differ significantly from classical aerobically functioning mitochondria. Mitochondrial ribosomal RNA sequences bear much more in common with bacteria than with ribosomes in the eukaryotic cytoplasm. For example, N-formylmethionyl transfer RNA has been found to exist only in mitochondria and bacteria. See, e.g., Yingying Le, Philip M. Murphy and Ji Ming Wang, Formyl-peptide receptors revisited; Trends in Immunology, 23, 11, 541-548; and Dyer, Betsey Dexter and Robert Obar (editors), 1985. The Origin of Eukaryotic Cells, Van Nostrand Reinhold Company, Inc., NY, each of which is incorporated by reference in its entirety.

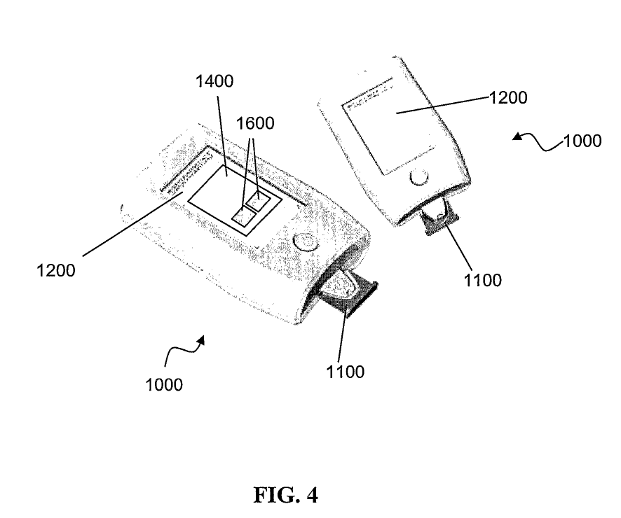

Referring to FIG. 4, reader instrument 1000 accepts test cartridge 1100 and includes display 1200. The display 1200 may be used to display images in various formats, for example, text, joint photographic experts group (JPEG) format, tagged image file format (TIFF), graphics interchange format (GIF), or bitmap. Display 1200 can also be used to display text messages, help messages, instructions, queries, test results, and various information to patients.

Display 1200 can provide a user with an input region 1400. Input region 1400 can include keys 1600. In one embodiment, input region 1400 can be implemented as symbols displayed on the display 1200, for example when display 1200 is a touch-sensitive screen. User instructions and queries are presented to the user on display 1200. The user can respond to the queries via the input region.

Reader 1000 also includes a cartridge reader, which accepts diagnostic test cartridges 1100 for reading. The cartridge reader can measure the level of an analyte based on, for example, the magnitude of an optical change, an electrical change, or other detectable change that occurs on a test cartridge 1100. For reading cartridges that produce an optical change in response to analyte, the cartridge reader can include optical systems for measuring the detectable change, for example, a light source, filter, and photon detector, e.g., a photodiode, photomultiplier, or Avalance photo diode. For reading cartridges that produce an electrical change in response to analyte, the cartridge reader can include electrical systems for measuring the detectable change, including, for example, a voltameter or amperometer.

Device 1000 further can include a communication port (not pictured). The communication port can be, for example, a connection to a telephone line or computer network. Device 1000 can communicate the results of a measurement to an output device, remote computer, or to a health care provider from a remote location.

A patient, health care provider, or other user can use reader 1000 for testing and recording the levels of various analytes, such as, for example, a biomarker, a metabolite, or a drug of abuse. Various implementations of diagnostic device 1000 may access programs and/or data stored on a storage medium (e.g., a hard disk drive (HDD), flash memory, video cassette recorder (VCR) tape or digital video disc (DVD); compact disc (CD); or floppy disk). Additionally, various implementations may access programs and/or data accessed stored on another computer system through a communication medium including a direct cable connection, a computer network, a wireless network, a satellite network, or the like.

The software controlling the reader can be in the form of a software application running on any processing device, such as, a general-purpose computing device, a personal digital assistant (PDA), a special-purpose computing device, a laptop computer, a handheld computer, or a network appliance.

The reader may be implemented using a hardware configuration including a processor, one or more input devices, one or more output devices, a computer-readable medium, and a computer memory device. The processor may be implemented using any computer processing device, such as, a general-purpose microprocessor or an application-specific integrated circuit (ASIC). The processor can be integrated with input/output (I/O) devices to provide a mechanism to receive sensor data and/or input data and to provide a mechanism to display or otherwise output queries and results to a service technician. Input device may include, for example, one or more of the following: a mouse, a keyboard, a touch-screen display, a button, a sensor, and a counter.

The display 1200 may be implemented using any output technology, including a liquid crystal display (LCD), a television, a printer, and a light emitting diode (LED). The computer-readable medium provides a mechanism for storing programs and data either on a fixed or removable medium. The computer-readable medium may be implemented using a conventional computer hard drive, or other removable medium. Finally, the system uses a computer memory device, such as a random access memory (RAM), to assist in operating the reader.

Implementations of the reader can include software that directs the user in using the device, stores the results of measurements. The reader 1000 can provide access to applications such as a medical records database or other systems used in the care of patients. In one example, the device connects to a medical records database via the communication port. Device 1000 may also have the ability to go online, integrating existing databases and linking other websites.

In general, the cartridge can be made by depositing reagents on a base and sealing a lid over the base. The base can be a micro-molded platform or a laminate platform.

Other embodiments are within the scope of the following claims.

1. A method for monitoring health of a mammalian subject comprising:
identifying a mitochondrial marker in a sample obtained from a subject; and
associating the mitochondrial marker with a status of health of the subject.

2. The method of claim 1, wherein the mitochondrial marker includes a nucleic acid.

3. The method of claim 1, wherein monitoring health includes detecting, screening, diagnosing, monitoring, or managing therapy of acute coronary syndromes.

4. The method of claim 1, wherein monitoring health includes detecting, screening, diagnosing, monitoring, or managing therapy of chronic angina.

5. The method of claim 1, wherein monitoring health includes detecting, screening, diagnosing, monitoring, or managing therapy of ischemia and the subject is in a patient suffering from heart failure.

6. The method of claim 1, wherein monitoring health includes detecting, screening, diagnosing, monitoring, or managing therapy of ischemia and the subject is in a patient suffering from stroke.

7. The method of claim 1, further comprising obtaining the sample from the patient before cardiac surgery, exercise treadmill, or pharmacologic stress testing.

8. The method of claim 1, further comprising obtaining the sample from the patient during cardiac surgery, exercise treadmill, or pharmacologic stress testing.

9. The method of claim 1, further comprising obtaining the sample from the patient after cardiac surgery, exercise treadmill, or pharmacologic stress testing.

10. The method of claim 1, wherein the sample is blood, plasma, or serum.

11. The method of claim 1, wherein the sample is blood.

12. The method of claim 1, wherein the mitochondrial marker is a mitochondrial DNA.

13. The method of claim 1, wherein the mitochondrial marker is a mitochondrial RNA.

14. The method of claim 1, wherein the mitochondrial marker is a polypeptide encoded by nuclear DNA.

15. The method of claim 1, wherein the mitochondrial marker is a subunit of NADH dehydrogenase, a subunit of cytochrome c oxidase, a subunits of F0F1ATPase, or cytochrome b.

16. The method of claim 1, wherein the mitochondrial marker is an FPR ligand.

17. The method of claim 1, wherein the mitochondrial marker is an N-formyl polypeptide.

18. The method of claim 1, wherein the mitochondrial marker is a caspase.

19. The method of claim 1, wherein the mitochondrial marker is a regulatory marker.

20. The method of claim 19, wherein the regulatory marker is cytochrome c, apoptosis-inducing factor, apoptotic protein activating factor-1, second mitochondria-derived activator of caspases, direct IAP-binding protein, serine protease omi/HtrA2, or endonuclease G.

21. The method of claim 1, wherein associating the mitochondrial marker with a status of health of the subject includes assessing the cardiac health of the subject.

22. The method of claim 1, wherein associating the mitochondrial marker with a status of health of the subject includes assessing the neurological health of the subject.

23. The method of claim 1, wherein associating the mitochondrial marker with a status of health of the subject includes assessing the pulmonary health of the subject.

24. The method of claim 1, wherein associating the mitochondrial marker with a status of health of the subject includes assessing a treatment protocol for the subject.

25. The method of claim 1, wherein identifying a mitochondrial marker includes determining a level of the mitochondrial marker in the sample.

26. The method of claim 1, further comprising identifying a necrosis marker in the sample.

27. The method of claim 26, wherein the necrosis marker is a troponin, CK-MB, myoglobin, or fatty acid binding protein.

28. The method of claim 1, further comprising identifying an ischemia marker in the sample.

29. The method of claim 28, wherein the ischemia maker is ischemia modified albumin, a fatty acid, whole blood choline, lipoprotein-associated phospholipase, or an oxidised lipid.

30. The method of claim 1, wherein identifying a mitochondrial marker in a sample obtained from a subject includes contacting the sample with a ligand for the mitochondrial marker.

31. The method of claim 30, wherein the ligand is an antibody, an antibody fragment, a modified antibody, chimeric antibody, soluble receptor, aptamer, or a nucleic acid capable of hybridizing to the mitochondrial marker under high stringency conditions.

32. The method of claim 30, wherein the ligand is a formyl peptide receptor, a formyl peptide receptor sub-unit, a fragment of a formyl peptide receptor, or an encoded sequence of binding region of a formyl peptide.

33. A system for monitoring health comprising:
a cartridge including a sample port and a first assay, wherein the first assay recognizes a mitochondrial marker; and
a cartridge reader including a detector configured to measure a level of the mitochondrial marker recognized by the assay.

34-57. (canceled)

58. A cartridge for monitoring health comprising:
a cartridge including a sample port and a first assay, wherein the first assay recognizes a mitochondrial marker.

59-80. (canceled)

81. A method of detecting ischemia comprising:
obtaining a sample from a subject suspected to have ischemia; and
assaying the sample for a mitochondrial marker.

82-87. (canceled)
